(19) United States
(12) Patent Application Publication
(10) Pub. No.: US 2023/0415137 A1
(43) Pub. Date: Dec. 28, 2023

(54) PHOTOCATALYST COMPOSITION, PHOTOCATALYST COMPOSITION SOLUTION, PHOTOCATALYST MEMBER, METHOD FOR USING PHOTOCATALYST COMPOSITION, AND SPACE DISINFECTION METHOD

(71) Applicant: YUSHIRO CHEMICAL INDUSTRY CO., LTD., Tokyo (JP)

(72) Inventors: Genta NAKAMURA, Koza-gun, Kanagawa (JP); Yasumasa OHASHI, Koza-gun, Kanagawa (JP)

(21) Appl. No.: 18/030,955

(22) PCT Filed: Sep. 22, 2021
(86) PCT No.: PCT/JP2021/034723

§ 371 Date: Apr. 7, 2023

(30) Foreign Application Priority Data
Oct. 15, 2020 (JP) 2020-174034
Aug. 10, 2021 (JP) 2021-130590

(51) Int. Cl. B01J 31/02; B01J 35/00; B01J 31/22

(52) U.S. Cl. CPC B01J 31/0244; B01J 35/004; B01J 31/0209; B01J 31/2213

(57) ABSTRACT

Provided are a photocatalyst composition that provides a strong photocatalytic effect with visible light, a photocatalyst composition solution using the photocatalyst composition, a photocatalyst member, a method for using the photocatalyst composition, and a space disinfection method. The photocatalyst composition contains a compound (A) having an isoalloxazine skeleton or an alloxazine skeleton and a sacrificial agent (B).

### TECHNICAL FIELD

The present invention relates to a photocatalyst composition, a photocatalyst composition solution, a photocatalyst member, a method for using the photocatalyst composition, and a space disinfection method.

### BACKGROUND ART

Photocatalysts that exhibit catalytic action when irradiated with light are known. Photocatalysts can easily decompose organic materials and perform bactericidal treatment simply when irradiated with light, and therefore various applications have been investigated.

Currently, titanium oxide is widely used as a photocatalyst. In order for titanium oxide to exhibit a catalytic action, it is necessary to irradiate titanium oxide with ultraviolet light, and titanium oxide has been pointed out to be carcinogenic, which may limit the application thereof. Thus, photocatalysts applicable to various uses have been investigated.

For example, Patent Literature 1 discloses a photocatalyst including, as an active component, a reaction product containing the Fe²⁺ complex of a reducing organic material, the reaction product obtained by mixing a reducing organic material consisting of ascorbic acid and a specific iron feedstock in a specific ratio in the presence of water under conditions of 40 to 100° C. for 10 seconds to 10 days.

In addition, Patent Literature 2 discloses a method for sterilizing a polymeric material, the method including: a step of providing the polymeric material with a radiosensitizer; and a step of applying suitable radiation at a dose and for a time effective to sterilize the polymeric material, and describes 200 μg/mL or more of riboflavin is used as the radiosensitizer.

### CITATION LIST

### Patent Literature

Patent Literature 1: Japanese Unexamined Patent Application Publication No. 2018-023977
Patent Literature 2: Published Japanese Translation of PCT International Publication for Patent Application, No. 2010-508403

### SUMMARY OF INVENTION

### Technical Problem

An object of the present invention is to provide a photocatalyst composition that provides a strong photocatalytic effect with visible light, a photocatalyst composition using the photocatalyst composition, a photocatalyst member, a method for using the photocatalyst composition, and a space disinfection method.

### Solution to Problem

The photocatalyst composition according to the present invention contains a compound (A) having an isoalloxazine skeleton or an alloxazine skeleton, and a sacrificial agent (B).

In the above photocatalyst composition, the compound (A) having an isoalloxazine skeleton or an alloxazine skeleton may include a compound represented by the general formula (1) below or a compound represented by the general formula (2) below.

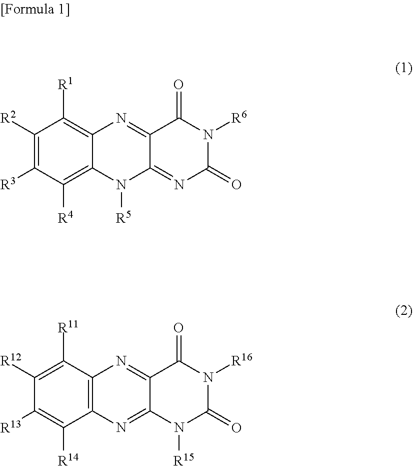

In the formula (1),

R¹, R², R³, and R⁴ each independently represent a hydrogen atom, a halogen atom, or a hydrocarbon group optionally having a substituent, and

R⁵ and R⁶ each independently represent a hydrogen atom or a hydrocarbon group optionally having a substituent.

In the formula (2),

R¹¹, R¹², R¹³, and R¹⁴ each independently represent a hydrogen atom, a halogen atom, or a hydrocarbon group optionally having a substituent, and

R¹⁵ and R¹⁶ each independently represent a hydrogen atom or a hydrocarbon group optionally having a substituent.

In the above photocatalyst composition, the compound (A) having an isoalloxazine skeleton or an alloxazine skeleton may include one or more selected from riboflavin and riboflavin derivatives.

In the above photocatalyst composition, the sacrificial agent (B) may include one or more selected from ascorbic acid compounds, polyols, polyphenols, and alkanolamines.

In the above photocatalyst composition, the sacrificial agent (B) may include one or more selected from chitosan and polyethyleneimine.

In the above photocatalyst composition, the chitosan may have a degree of deacetylation of 50% or more.

In the above photocatalyst composition, the chitosan may have a molecular weight of 5,000 to 1,000,000.

The above photocatalyst composition may further contain one or more ions (C) selected from iron ions, copper ions, and nickel ions.

In the above photocatalyst composition, the mass ratio (A/B) of the compound (A) having an isoalloxazine skeleton or an alloxazine skeleton to the sacrificial agent (B) may be 1/2 to 1/10000.

The present invention provides a photocatalyst composition solution containing the above photocatalyst composition and a solvent.

The present invention provides a photocatalyst member including a coating film containing the above photocatalyst composition on a base material.

A method for using the photocatalyst composition of the present invention may be a method including:
supplying a sacrificial agent (B) to
(I) a composition solution containing a compound (A) having an isoalloxazine skeleton or an alloxazine skeleton and a solvent, or
(II) a member including a coating film containing the compound (A) having an isoalloxazine skeleton or an alloxazine skeleton on a base material.

The present invention further provides a space disinfection method including spraying the above photocatalyst composition into a space.

### Advantageous Effects of Invention

The present invention provides a photocatalyst composition that provides a strong photocatalytic effect with visible light, a photocatalyst composition using the photocatalyst composition, a photocatalyst member, a method for using the photocatalyst composition, and a space disinfection method.

### BRIEF DESCRIPTION OF THE DRAWING

FIG. 1 shows results of the durability test.

### DESCRIPTION OF EMBODIMENTS

Hereinafter, the photocatalyst composition according to the present invention (hereinafter also referred to as the present photocatalyst composition), a photocatalyst composition using the photocatalyst composition, a photocatalyst member, a method for using the photocatalyst composition, and a space disinfection method will be described in order.

In addition, “to” between two numerical values indicating a numerical range includes the lower limit and upper limit unless otherwise specified.

### [Photocatalyst Composition]

The present photocatalyst composition contains a compound (A) having an isoalloxazine skeleton or an alloxazine skeleton (hereinafter simply referred to as compound (A)) and a sacrificial agent (B).

In the present photocatalyst composition, the compound (A) having an isoalloxazine skeleton or an alloxazine skeleton functions as a photosensitizer, and electrons of the sacrificial agent (B) are supplied to molecular oxygen via an excited photosensitizer to generate active oxygen, thereby decomposing organic materials and performing bactericidal actions. The effect of obtaining a strong photocatalytic effect with visible light by the combination of the above compounds is presumed as follows.

It is presumed that a compound having the compound (A) efficiently absorbs visible light (for example, wavelength of 400 to 600 nm), undergoes a redox reaction with the sacrificial agent (B), and is thus reduced. It is presumed that the reduced compound (A) causes a redox reaction with oxygen in the air to be oxidized, thereby generating H₂O₂. Thus, the present photocatalyst composition can generate H₂O₂ from visible light, and can provide a strong photocatalytic effect.

The photocatalyst composition of the present invention provides an excellent photocatalytic effect despite of using a light source with relatively low output such as a white LED. The present photocatalyst composition can provide a strong photocatalytic effect despite of using the compound (A) at a relatively low concentration, and therefore can be used, for example, while suppressing the color as a solution.

In the present photocatalyst composition, only the sacrificial agent (B) is consumed to provide the photocatalytic effect, and the compound (A) is preserved. Therefore, the photocatalytic effect can be easily reproduced by supplying the sacrificial agent (B).

Furthermore, the present photocatalyst composition is soluble in water and is highly safe when taken into the living body of humans and the like, and thus can be used in various applications.

The present photocatalyst composition may contain at least the compound (A) and the sacrificial agent (B), and may further contain other components as long as the effect of the present invention is exhibited. Each component that can be contained in the present photocatalyst composition is described below.

### <Compound (A) Having Isoalloxazine Skeleton or Alloxazine Skeleton>

The compound (A) functions as a photosensitizer in which the isoalloxazine skeleton or alloxazine skeleton absorbs visible light and the like.

In the present photocatalyst composition, from the viewpoint of obtaining an excellent photocatalytic action, the compound (A) is preferably a compound represented by the general formula (1) below or a compound represented by the general formula (2) below.

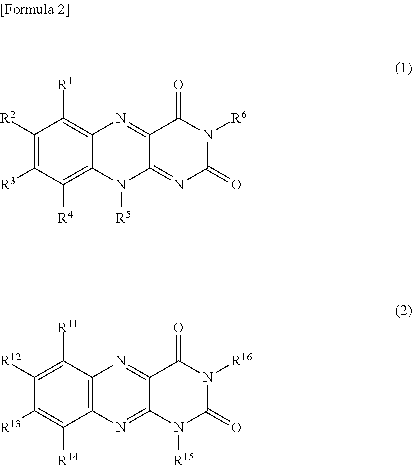

In the formula (1),

R¹, R², R³, and R⁴ each independently represent a hydrogen atom, a halogen atom, or a hydrocarbon group optionally having a substituent, and

R⁵ and R⁶ each independently represent a hydrogen atom or a hydrocarbon group optionally having a substituent.

In the formula (2),

R¹¹, R¹², R¹³, and R¹⁴ each independently represent a hydrogen atom, a halogen atom, or a hydrocarbon group optionally having a substituent, and

R¹⁵ and R¹⁶ each independently represent a hydrogen atom or a hydrocarbon group optionally having a substituent.

Examples of the halogen atom for R¹ to R⁴ and R¹¹ to R¹⁴ include a fluorine atom, a chlorine atom, and a bromine atom.

For R¹ to R⁴ and R¹¹ to R¹⁴, examples of the hydrocarbon group include a linear or branched alkyl group having 1 to 6 carbon atoms, and a cycloalkyl group or aryl group having 6 to 12 carbon atoms and optionally having a linear or branched alkyl group as a substituent. Examples of the linear or branched alkyl group include a methyl group, an ethyl group, a n-butyl group, a tert-butyl group, and a hexyl group. Examples of the cycloalkyl group include a cyclohexyl group. Examples of the aryl group include a phenyl group and a naphthyl group. Examples of the substituent that the hydrocarbon group in R¹ to R⁴ and R¹¹ to R¹⁴ may have include a hydroxy group, a carboxy group, a halogen atom, and a phosphoric acid group.

From the viewpoint of photocatalytic activity, each of R¹, R⁴, R¹¹, and R¹⁴ preferably independently represents a hydrogen atom. From the viewpoint of photocatalytic activity, each of R², R³, R¹², and R¹³ preferably independently represents an unsubstituted linear or branched alkyl group, more preferably an alkyl group having 1 to 4 carbon atoms, and still more preferably a methyl group.

Examples of the hydrocarbon group for R⁶, R¹⁵, and R¹⁶ include the same groups as those for R¹ and the like, and preferable embodiments are also the same. From the viewpoint of photocatalytic activity, each of R⁶, R¹⁵, and R¹⁶ preferably independently represents a hydrogen atom.

Examples of the hydrocarbon group for R⁵ include the same groups as those for R¹ and the like. Examples of the substituent that the hydrocarbon group in R⁵ may have include a hydroxy group or an ester thereof, and a phosphate ester. Among them, the substituent in R⁵ is preferably a ribityl group (—CH₂—CHOH—CHOH—CHOH—CH₂OH), an esterified ribityl group, or a phosphoric acid-esterified ribityl group.

Furthermore, the compound (A) is particularly preferably riboflavin or a riboflavin derivative. Riboflavin, also known as vitamin B2, is highly safe and can be suitably used, for example, in applications in which it may be taken into the body of humans or other organisms.

Specific examples of the riboflavin derivative include riboflavin tetrabutyrate, riboflavin tetraacetate, lumichrome (7,8-dimethylalloxazine), riboflavin phosphate and a salt thereof (for example, sodium salt and potassium salt), and flavin adenine dinucleotide.

Among them, riboflavin and riboflavin derivatives are preferably riboflavin, riboflavin tetrabutyrate, lumichrome, and riboflavin sodium phosphate, from the viewpoints of photocatalytic activity, safety, industrial availability, and the like.

In the present photocatalyst composition, the compound (A) may be used singly or in combination of two or more.

### <Sacrificial Agent (B)>

In the present photocatalyst composition, the sacrificial agent (B) refers to a compound that proceeds the reaction while the compound itself is decomposed in the photocatalytic reaction. The use of the sacrificial agent (B) in combination with the compound (A) can provide an excellent photocatalytic effect with visible light.

As the sacrificial agent (B), from the viewpoints of photocatalytic activity, safety, industrial availability, and the like, ascorbic acid compounds, polyols, polyphenols, alkanolamines, chitosan, and polyethyleneimine are preferable. Of these, the sacrificial agent may be used singly or in combination of two or more.

Examples of the ascorbic acid compounds include ascorbic acid, and isoascorbic acid (erythorbic acid) and a salt thereof. The counter ion of the salt is not particularly limited, and examples thereof include a lithium ion, a sodium ion, a potassium ion, a calcium ion, a magnesium ion, and an iron ion.

Examples of the polyols include compounds having two or more hydroxy groups in one molecule, preferably three or more hydroxy groups in one molecule. Among them, a polyol satisfying the composition formula CₙH₂ₙ₋₂Oₙ (in which n is an integer of 2 or more) and a polyether thereof are preferable, from the viewpoint of high electricity-donating ability. Specific examples of the polyol satisfying the composition formula include glycerin, threitol, arabitol, xylitol, and ribitol, and examples of the polyether thereof include polyglycerin. In addition, polyols that are liquid at the operating temperature (for example, 25° C.) can also function as a solvent, which will be described later.

Examples of polyphenols include a compound having two or more phenolic hydroxyl groups in one molecule, and the hydroxyl group may be in the form of a salt. As polyphenols, naturally derived polyphenols are preferable from the viewpoints of having a low environmental load and being highly safe to the living body. Specific examples thereof include ferulic acid, tannic acid, catechin acid, epigallocatechin gallate, gallic acid, chlorogenic acid, caffeic acid, ellagic acid, curcumin, lignans, rutin, hesperidin, kaempferol, quercetin, isoflavone, anthocyanin, theaflavin, theaflavin gallate, cyanidin, resveratrol, chalconic acid, ellagic acid, quercitrin, isoquercitrin, myricitrin, myricetin, delphinidin, delphine, nasunin, peonidin, peonin, neohesperidin, hesperetin, naringin, lingenin, purnin, astragalin, apiin, apigeni, daidzein, daidzin, glycitein, glycitin, genistein, genistin, malvidin, malvin, enine, leucocyanidin, cyanine, chrysanthemine, keracyanin, ideine, mecocyanin, pelargonidin, callistefin, p-coumaric acid, sesamin, sesaminol, sesamolin and a sodium salt thereof, a potassium salt thereof, and a calcium salt thereof. These may be used singly or in combination of two or more.

Examples of the alkanolamines include a compound having one or more hydroxy groups and one or more amino groups in one molecule, and a compound obtained by salt-forming the amino group of the compound with an acid such as an organic acid or an oxo acid. Specific examples of the alkanolamines include triethanolamine, diethanolamine, ethanolamine, methanolamine, triisopropanolamine, diisopropanolamine, isopropanolamine, butylethanolamine, 2,2′-(cyclohexylimino)bisethanol, N-tert-butyldiethanolamine, dimethylaminoethanol, N-piperidine ethanol, N-morpholine ethanol, 2-(methylamino)ethanol, and 2-[(hydroxymethyl)amino]ethanol.

Examples of the acid that forms a salt with alkanolamines include: an organic acid such as acetic acid, lactic acid, succinic acid, salicylic acid, citric acid, gluconic acid, tartaric acid, lactic acid, fumaric acid, malic acid, phosphoric acid, ascorbic acid, erythorbic acid, malic acid, oleic acid, phytic acid, and octylphosphonic acid; and an oxoacid such as phosphoric acid, boric acid, sulfuric acid, nitric acid, polyphosphoric acid, and silicic acid.

The present inventors have further found that selecting chitosan or polyethyleneimine as the sacrificial agent provides excellent long-term stability of the photocatalytic activity. In addition, these polymers can maintain the skeleton after losing activity as a sacrificial agent of the present photocatalyst composition, and can function, for example, as a binder component.

In the present disclosure, chitosan is a compound that has two or more glucosamine-derived structural units, may have an acetylglucosamine-derived structural unit, and may have other structures. Chitosan in the present disclosure may also be a relatively low molecular weight compound that can be classified as a chitosan oligosaccharide. From the viewpoint of photocatalytic activity, the chitosan is preferably chitosan(poly-1,4-β-D-glucosamine) obtained by bonding and polymerizing the 1st and 4th positions of a plurality of D-glucosamines or chitosan oligosaccharide. Chitosan may be polyglucosamine consisting only of a structural unit derived from glucosamine, or may have a structural unit derived from N-acetylglucosamine.

The chitosan can be obtained, for example, by deacetylating chitin obtained from shrimps, crabs, mushrooms, insects, cell walls of fungi, and the like with alkali. The degree of deacetylation of chitosan is preferably 50% or more, more preferably 70% or more, still more preferably 80% or more, and particularly preferably 90% or more, from the viewpoint of the photocatalytic effect and durability of the photocatalytic effect.

The degree of deacetylation of chitosan is represented by [number of structural units derived from glucosamine/(number of structural units derived from glucosamine+number of structural units derived from acetylglucosamine)]×100 (%), and can be measured, for example, by a colloid titration method using polyvinyl potassium sulfate (PVSK).

In the present invention, chitosan may include a chitosan derivative. Examples of the chitosan derivative include hydroxyalkylene chitosan such as hydroxyethyl chitosan obtained by reacting the above chitosan with an alkylene oxide such as ethylene oxide and propylene oxide;
carboxymethylated chitosan obtained by reacting the above chitosan with (1) monochloroacetic acid or reductive amination of (2) glyoxalic acid; and
chitosan having a crosslinked structure obtained by reacting the above chitosan with a dichloride such as adipic acid dichloride.

The molecular weight of chitosan is preferably 350 or more, more preferably 5,000 or more, still more preferably 30,000 or more, and particularly preferably 40,000 or more, from the viewpoint of the photocatalytic effect. Meanwhile, the molecular weight of chitosan is preferably 1,000,000 or less, more preferably 500,000 or less, and still more preferably 200,000 or less, from the viewpoints of deterioration of the photocatalytic effect due to a decrease in the mobility of chitosan molecules and deterioration of handleability due to an increase in viscosity.

In the present invention, the molecular weight is a peak top molecular weight (Mp) obtained as a standard pullulan conversion value by aqueous gel filtration chromatography (Chromaster (register trade mark), manufactured by Hitachi High-Tech Science Corporation).

Chitosan may be extracted from natural products or synthesized as described above. Commercially available polyglucosamine may also be used.

A polyethyleneimine is a compound having one or more types of structural units selected from a structural unit represented by —CH₂—CH₂—NH— and a structural unit represented by —CH₂—CH₂—N< and having two or more pieces of the structural units in one molecule.

Polyethyleneimine may have a structural unit other than the structural unit represented by —CH₂—CH₂—NH— or —CH₂—CH₂—N<.

The proportion of the structural unit represented by —CH₂—CH₂—NH— or —CH₂—CH₂—N< in polyethyleneimine is preferably 50% by mass or more, more preferably 70% by mass or more, and still more preferably 80% by mass or more, relative to the total mass of polyethyleneimine.

In the present invention, polyethyleneimine may include a polyethyleneimine derivative. Examples of the polyethyleneimine derivative include hydroxyalkylene polyethyleneimine such as hydroxyethyl polyethyleneimine obtained by reacting the above polyethyleneimine with an alkylene oxide such as ethylene oxide and propylene oxide;
carboxymethylated polyethyleneimine obtained by reacting the above polyethyleneimine with (1) monochloroacetic acid or reductive amination of (2) glyoxalic acid; and
polyethyleneimine having a crosslinked structure obtained by reacting the above polyethyleneimine with a dichloride such as adipic acid dichloride.

The peak top molecular weight (Mp) of polyethyleneimine is preferably 1,000 or more, more preferably 10,000 or more, and still more preferably 25,000 or more, from the viewpoint of the photocatalytic effect. Meanwhile, the weight average molecular weight of polyethyleneimine is preferably 1,000,000 or less, more preferably 800,000 or less, and still more preferably 500,000 or less, from the viewpoint of ease of synthesis.

Polyethyleneimine can be synthesized, for example, by ring-opening polymerization of aziridine. Commercially available polyethyleneimine may also be used.

Any one of chitosan and polyethyleneimine may be used in the present photocatalyst composition, or chitosan and polyethyleneimine may be used in combination.

In the present photocatalyst composition, the mass ratio (A/B) of the compound (A) to the sacrificial agent (B) is preferably 1/2 to 1/10000. A/B of 1/10000 or more provides excellent photocatalytic effect. Meanwhile, A/B of 1/2 or less exhibits a sufficient photocatalytic effect. Particularly, A/B is preferably 1/5 to 1/200, more preferably 1/10 to 1/200. The sacrificial agent (B) decreases in an amount during use, and thus may be added excessively previously. When the amount of the sacrificial agent (B) is reduced, the sacrificial agent (B) may be newly added.

### <Other Components>

The present photocatalyst composition may further contain other components as long as the effect of the present invention is exhibited. Other components include each component exemplified in the photocatalyst composition solution and photocatalyst member described below, and various additives.

Furthermore, the present photocatalyst composition preferably contains one or more ions (C) selected from iron ions, copper ions, and nickel ions. In the photocatalyst composition containing the ion (C), it is presumed that H₂O₂ generated by visible light undergoes a redox reaction with the ion (C) and is reduced to generate a OH radical. Thus, the present photocatalyst composition can generate a highly reactive OH radical from visible light, and can provide a strong photocatalytic effect.

The above ion (C) may be used singly or in combination of two or more. The present photocatalyst composition preferably contains iron ions or copper ions, and more preferably contains iron ions, from the viewpoint of photocatalytic activity and use in various applications.

For the above ion (C), for example, ions contained in the water source (for example, well water, river/lake water, seawater, tap water, agricultural water, industrial water and the like) when using water as a solvent described below may be utilized, ions contained in raw materials and other environments may be utilized, and metal ions that can enter in the process for producing the photocatalyst composition may be utilized.

In addition, by using the following ion source compounds in combination, the amount of ions in the present photocatalyst composition can be easily adjusted.

### <Metal Ion Source>

In order to use the ion (C), the photocatalyst composition preferably contains a metal ion source selected from an iron ion source, a copper ion source, and a nickel ion source. The iron ion source, copper ion source, and nickel ion source are compounds that can produce iron ions, copper ions, and nickel ions, respectively, and the produced metal ions can efficiently generate OH radicals.

Examples of the source of iron ions include dissociable iron compounds. The iron compound may contain divalent iron ions or may contain trivalent iron ions. This is because the iron ion alternates between a divalent state and a trivalent state in the photocatalytic reaction.

Examples of the iron compound include iron(II) chloride, iron(II) sulfate, iron(II) nitrate, iron(II) hydroxide, iron(II) oxide, iron(II) acetate, iron(II) lactate, sodium iron(II) citrate, iron (II) gluconate, iron (II) carbonate, iron (II) fumarate, iron(III) chloride, iron(III) sulfate, iron(III) citrate, iron(III) ammonium citrate, iron(III) EDTA, iron(III) oxide, iron(III) nitrate, iron(III) hydroxide, iron (III) pyrophosphate, iron (II) ascorbate, iron (II) chlorophyllin sodium, iron (II) chlorophyll, and iron (II) acetylacetonate.

Examples of the source of copper ions include dissociable copper compounds. The copper compound may contain monovalent copper ions or may contain divalent copper ions.

Examples of the copper compound include copper(II) sulfate, copper(II) chloride, copper(II) bromide, copper (I) iodide, copper (II) acetate, copper (II) nitrate, copper(II) citrate, copper(II) gluconate, copper(II) chlorophyllin sodium, copper (II) chlorophyll, and copper (II) acetylacetonate.

Examples of the source of nickel ions include dissociable nickel compounds. The nickel compound may contain divalent nickel ions or may contain trivalent nickel ions.

Examples of the nickel compound include nickel (II) sulfate, nickel (II) chloride, nickel (II) acetate, nickel (II) benzoate, and nickel (II) acetylacetonate.

The mass ratio (C/B) of the mass of the metal ion source (C) to the mass of the sacrificial agent (B) is preferably 1/100000 to 1/10, more preferably 1/10000 to 1/200, still more preferably 1/5000 to 1/20, from the viewpoint of excellent photocatalytic effect.

When a metal ion source is used, the present photocatalyst composition preferably contains water to such an extent that the ion source compound dissociates during the photocatalytic reaction.

### [Photocatalyst Composition Solution]

The present photocatalyst composition solution contains the above photocatalyst composition and a solvent.

The solvent can be appropriately selected and used from solvents capable of dissolving or dispersing each component of the above photocatalyst composition. From the viewpoint of allowing to dissolve each component of the photocatalyst composition, the solvent preferably contains one or more solvents selected from water and water-soluble solvents, and more preferably contains water. Examples of the water-soluble solvent include an alcohol-based solvent such as ethanol, isopropanol, ethylene glycol, propylene glycol, glycerin, dipropylene glycol, and diethylene glycol.

When a mixed solvent of water and a water-soluble solvent is used, the water-soluble solvent is preferably 10% by mass or less, preferably 5% by mass or less, in 100% by mass of the solvent. Furthermore, the solvent is preferably a solvent consisting essentially of water, from the viewpoint of being applicable to various applications.

The proportion of the compound (A) in the present photocatalyst composition solution is preferably 1 to 100 μg/mL, more preferably 2 to 80 μg/mL, still more preferably 3 to 50 μg/mL, and particularly preferably 4 to 20 μg/mL, based on the volume of the solution. Setting the proportion to the lower limit or more exhibits excellent photocatalytic performance. Meanwhile, setting the proportion to the upper limit or less can provide a photocatalyst composition solution with coloration suppressed. The use of a photocatalyst composition solution to be colored is problematic in that the light transmission becomes insufficient, and the photocatalytic effect may not be obtained at a position away from the light source. The present photocatalyst composition solution suppresses coloration, thereby allowing to suppress occurrence of the problem of the insufficient light transmission, even if used in a large amount. In addition, this is suitable for visual recognition of the inside of the photocatalyst composition solution.

The proportion of the sacrificial agent (B) in the present photocatalyst composition solution is preferably 5 to 2000 μg/mL, more preferably 25 to 1500 μg/mL, and still more preferably 50 to 1200 μg/mL, based on the volume of the solution. Setting the amount within the above range can provide a solution with excellent photocatalytic effect. Since the sacrificial agent (B) is consumed, the amount may be temporarily out of the above range. When the proportion of the sacrificial agent (B) becomes low, the sacrificial agent (B) may be added as appropriate.

When the present photocatalyst composition solution contains the above metal ion source, the total proportion of the specific metal ion source in the present photocatalyst composition solution is preferably 0.01 to 10 μg/mL, more preferably 0.02 to 5 μg/mL, and still more preferably 0.1 to 2 μg/mL, based on the volume of the solution. Setting the total proportion to 0.01 μg/mL or more provides the solution that sufficiently exhibits the effect of the specific metal ion and becomes excellent in photocatalytic effect.

The present photocatalyst composition solution may further contain other components as long as the effect of the present invention is exhibited. Examples of other components include surfactants, antifoaming agents, preservatives, and pH adjusters.

Surfactants are used for the purpose of imparting wettability to base materials and the like, and can be appropriately selected and used from known surfactants such as anionic surfactants, nonionic surfactants, and cationic surfactants, depending on the application.

The present photocatalyst composition solution can be applied to all conventionally known uses in which photocatalysts are used. Furthermore, the present photocatalyst composition solution is highly safe even if taken into the body of a human or an organism, and thus is used for, for example, purification of the water in the tanks of air purifiers and humidifiers; space disinfection use by spraying in a space such as a room; water in aquariums for rearing aquatic organisms; water for growing plants; water-soluble metalworking fluids; food freshness-preserving liquids; deodorant liquids; disinfectant liquids; antiviral solutions; and cleaning liquids.

### [Photocatalyst Member]

The present photocatalyst member is a member including a coating film containing the present photocatalyst composition on a base material.

The base material may be appropriately selected from base materials such as resins, non-woven fabrics, metals, glass, and ceramics, depending on the application of the member. When a resin or the like that may deteriorate due to photocatalytic effect is used as the base material, a silicone coating film or the like may be provided as an undercoat layer before forming the coating film of the photocatalyst composition.

Of the sacrificial agents (B) in the present photocatalyst composition, chitosan and polyethyleneimine also function as binders, and have excellent adhesion to the base material when formed into a coating film.

The coating film of the photocatalyst composition may further contain other components as long as the effect of the present invention is exhibited. Other components may include each component exemplified in the above photocatalyst composition solution and a binder resin other than chitosan and polyethyleneimine.

Adhesion to the base material is further improved by using a binder resin. As the binder resin used in the present photocatalyst member, a water-based organic resin is preferable. Specific examples thereof include water-based urethane resins, water-based acrylic resins, water-based polyester resins, water-based epoxy resins, water-based fluorine resins, water-based silicone resins, and water-based polyethylene resins.

The coating film of the photocatalyst composition can be formed by a known coating method such as spraying the present photocatalyst composition, the present photocatalyst composition solution, or a mixture of these and the binder resin onto the surface of the base material.

The present photocatalyst member can be suitably used, for example, as building materials such as exterior walls of buildings, window glass, indoor flooring, and wallpaper, and filters having photocatalytic performance with porous base materials.

### [Method for Using Photocatalyst Composition]

An example of the method for using the present photocatalyst composition includes a method including:
supplying the sacrificial agent (B) to
(I) a composition solution containing a compound (A) having an isoalloxazine skeleton or an alloxazine skeleton and a solvent, or
(II) a member including a coating film containing the compound (A) having an isoalloxazine skeleton or an alloxazine skeleton on a base material.

According to the above use method, the sacrificial agent (B) is resupplied to the composition solution or member in which the sacrificial agent (B) has been reduced, allowing the photocatalytic performance to be easily regenerated. Examples of the supply method include, in the case of a solution, a method of adding a solution of chitosan or polyethyleneimine. In addition, in the case of a member, examples thereof include a method of spraying a solution of chitosan or polyethyleneimine onto the coating film.

As described above, by combining the compound (A) having an isoalloxazine skeleton or an alloxazine skeleton with the sacrificial agent (B), the present photocatalyst composition absorbs visible light and exhibits an excellent photocatalytic effect at a low concentration. The present photocatalyst composition can be used at a low concentration, and thus can be used, for example, in the form of an aqueous solution with suppressed coloration. In addition, the present photocatalyst composition has each component being excellent in safety to organisms, and thus can be suitably used for applications in which it is probably taken into the organism.

### EXAMPLES

The present invention will be specifically described below with reference to Examples and Comparative Examples, but the present invention is not limited to these Examples. In addition, hereinafter, “ppm” represents “μg/mL”.

### (First Example Group)

The first example group shows the effect of a photocatalyst composition in which the sacrificial agent (B) contains one or more selected from ascorbic acid compounds, polyols, polyphenols, and alkanolamines.

### Example 1: Preparation of Photocatalyst Composition Solution

Water was added to 10 parts by mass of riboflavin, 100 parts by mass of sodium L-ascorbate, and 10 parts by mass of iron (II) gluconate to prepare the concentration shown in Table 1, and the photocatalyst composition solution of Example 1 was obtained.

### Examples 2 to 18: Preparation of Photocatalyst Composition Solution

Photocatalyst composition solutions of Examples 2 to 18 were obtained in the same manner as in Example 1, except that the concentrations of each component and the blending amount in Example 1 were changed to those shown in Tables 1 to 3.

### Comparative Examples 1 to 13: Preparation of Solution

The solutions of Comparative Examples 1 to 13 were obtained in the same manner as in Example 1, except that the concentrations of each component and the blending amount in Example 1 were changed to those shown in Tables 1 to 3.

The “ascorbate iron complex” in the table was synthesized according to the description in Example 3 of Japanese Unexamined Patent Application Publication No. 2018-023977 (Patent Literature 1 above).

### [Evaluation]

### <Bactericidal Evaluation>

The indigenous bacteria collected from the ultrasonic humidifier is adjusted to a predetermined concentration, then was added to an autoclave-sterilized normal bouillon medium (Eiken (register trade mark), manufactured by Eiken Chemical Co., Ltd.). Shaking culture for 24 hours was performed in a shaking incubator at 30° C. and 120 rpm. The solution when the number of bacteria reached about 1.5×10⁹ CFU/ml was used as an inoculum solution.

Separately, the photocatalyst composition solutions obtained in the above Examples and Comparative Examples were also autoclave sterilized.

The inoculum solution was diluted with each of the photocatalyst composition solutions of the above Examples and Comparative Examples to prepare a solution having the number of bacteria of 1.5×10⁷ CFU/ml, which was used as a test solution.

Two similar samples for each example and comparative example were prepared and stored under the following two conditions. The number of bacteria was measured using “Sani-Check BF” (manufactured by Biosan Laboratories, Inc.) and evaluation was performed according to the following evaluation criteria. The results are shown in Tables 1 to 3.
(1) Using a white LED (FHF32W2, manufactured by Sanyo Electric Co., Ltd.) as a light source at 25° C., irradiation was performed for 2 hours at a photon flux density of 20.25 μmol m⁻²·s⁻¹.
(2) The sample was stored in a dark place for 2 hours at 25° C.

### (Evaluation Criteria)

A (outstanding): The number of bacteria was 0 to less than 1.0×10³ CFU/ml.
B (excellent): The number of bacteria was 1.0×10³ CFU/ml or more and less than 1.0×10⁴ CFU/ml.
C (good): The number of bacteria was 1.0×10⁴ CFU/ml or more and less than 1.0×10⁶ CFU/ml.
E (bad): The number of bacteria was 1.0×10⁶ CFU/ml or more and less than 1.5×10⁷ CFU/ml.

### <Colorability Evaluation>

An evaluation sample was obtained in such a manner that a predetermined amount of each photocatalyst composition obtained in Examples and Comparative Examples in a spray bottle was sprayed on a standard test cloth cut to 5 cm×5 cm from a position 10 cm away, and the color difference represented by the following formula before and after coating was measured. The results are shown in Tables 1 to 3.

Standard cloth: Crecia Techno Wipe C100-M, manufactured by Nippon Paper Crecia Co., Ltd.

Color difference ΔE=[(ΔL)²+(Δa)²+(Δb)²]^{1/2}
in which ΔL=L value (after coating)−L value (before coating)
Δa=a value (after coating)−a value (before coating)
Δb=b value (after coating)−b value (before coating)

TABLE 1

| | | Example | Example | Example | Example | Comparative | Comparative |
|---|---|---|---|---|---|---|---|
| | | 1 | 2 | 3 | 4 | Example 1 | Example 2 |
| Compound | Riboflavin | 10 ppm | | | | | |
| (A) | Riboflavin sodium phosphate | | 10 ppm | | | | 10 ppm |
| | Riboflavin butyrate | | | 10 ppm | | | |
| | Lumichrome | | | | 10 ppm | | |
| Sacrificial | Potassium L-ascorbate | | 100 ppm | | 100 ppm | | |
| agent | Sodium L-ascorbate | 100 ppm | | 100 ppm | | 100 ppm | |
| (B) | Calcium L-ascorbate | | | | | | |
| | L-ascorbic acid | | | | | | |
| | Sodium erythorbate | | | | | | |
| | Triethanolamine | | | | | | |
| | Triisopropanolamine | | | | | | |
| | Sodium ferulate | | | | | | |
| | Sodium tannate | | | | | | |
| | (+)-Catechin sodium | | | | | | |
| | (−)-Epigallocatechin sodium gallate | | | | | | |
| | Glycerin | | | | | | |
| | Polyglycerin | | | | | | |
| | Xylitol | | | | | | |
| Other | Alanine | | | | | | |
| components | Sodium glutamate | | | | | | |
| | Sodium itaconate | | | | | | |
| | Sodium citrate | | | | | | |
| Ion (C) | Iron(II) gluconate | 10 ppm | | | | | |
| (ion source) | Iron(II) citrate | | 5 ppm | | | 5 ppm | 5 ppm |
| | Iron(II) sulfate | | | 5 ppm | | | |
| | Iron(III) sulfate | | | | 5 ppm | | |
| Other | Iron ascorbate complex | | | | | | |
| components | Titanium oxide | | | | | | |
| Solvent | Propylene glycol/water = 1/1 mixture | | | | | | |
| | Purified water | Balance | Balance | Balance | Balance | Balance | Balance |
| Total | Total (%) | 100 | 100 | 100 | 100 | 100 | 100 |
| Evaluation | (1) Bactericidal activity after light irradiation | A | A | A | A | E | E |
| | (2) Bactericidal activity before light irradiation | E | E | E | E | E | E |
| | Colorability (color difference change ΔE) | 3.69 | 3.55 | 3.62 | 3.47 | 1.22 | 3.38 |

| | | Example | Comparative | Comparative | Comparative |
|---|---|---|---|---|---|
| | | 5 | Example 3 | Example 4 | Example 5 |
| Compound | Riboflavin | | | | |
| (A) | Riboflavin sodium phosphate | 10 ppm | | | |
| | Riboflavin butyrate | | | | |
| | Lumichrome | | | | |
| Sacrificial | Potassium L-ascorbate | | | | |
| agent | Sodium L-ascorbate | 100 ppm | | | |
| (B) | Calcium L-ascorbate | | | | |
| | L-ascorbic acid | | | | 480 ppm |
| | Sodium erythorbate | | | | |
| | Triethanolamine | | | | |
| | Triisopropanolamine | | | | |
| | Sodium ferulate | | | | |
| | Sodium tannate | | | | |
| | (+)-Catechin sodium | | | | |
| | (−)-Epigallocatechin sodium gallate | | | | |
| | Glycerin | | | | |
| | Polyglycerin | | | | |
| | Xylitol | | | | |
| Other | Alanine | | | | |
| components | Sodium glutamate | | | | |
| | Sodium itaconate | | | | |
| | Sodium citrate | | | | |
| Ion (C) | Iron(II) gluconate | | | | |
| (ion source) | Iron(II) citrate | | | | 20 ppm |
| | Iron(II) sulfate | | | | |
| | Iron(III) sulfate | | | | |
| Other | Iron ascorbate complex | | 1000 ppm | 10000 ppm | |
| components | Titanium oxide | | | | |
| Solvent | Propylene glycol/water = 1/1 mixture | | | | |
| | Purified water | Balance | Balance | Balance | Balance |
| Total | Total (%) | 100 | 100 | 100 | 100 |
| Evaluation | (1) Bactericidal activity after light irradiation | C | E | C | E |
| | (2) Bactericidal activity before light irradiation | E | E | C | E |
| | Colorability (color difference change ΔE) | 3.36 | 2.31 | 2.94 | 1.53 |

TABLE 2

| | | Example | Example | Example | Example | Example | Comparative | Comparative |
|---|---|---|---|---|---|---|---|---|
| | | 6 | 7 | 8 | 9 | 10 | Example 6 | Example 7 |
| Compound | Ribuflavin | | 10 ppm | 8 ppm | | | | |
| (A) | Riboflavin sodium phosphate | 10 ppm | | | 8 ppm | 8 ppm | | 10 ppm |
| | Riboflavin butyrate | | | | | | | |
| | Lumichrome | | | | | | | |
| Sacrificial | Potassium L-ascorbate | | | | | | | |
| agent | Sodium L-ascorbate | | | | | | | |
| (B) | Calcium L-ascorbate | 100 ppm | | | | | | |
| | L-ascorbic acid | | 100 ppm | | | | | |
| | Sodium erythorbate | | | 100 ppm | | | | |
| | Triethanolamine | | | | 100 ppm | | 100 ppm | |
| | Triisopropanolamine | | | | | 100 ppm | | |
| | Sodium ferulate | | | | | | | |
| | Sodium tannate | | | | | | | |
| | (+)-Catechin sodium | | | | | | | |
| | (−)-Epigallocatechin sodium gallate | | | | | | | |
| | Glycerin | | | | | | | |
| | Polyglycerin | | | | | | | |
| | Xylitol | | | | | | | |
| Other | Alanine | | | | | | | 100 ppm |
| components | Sodium glutamate | | | | | | | |
| | Sodium itaconate | | | | | | | |
| | Sodium citrate | | | | | | | |
| Jon (C) | Iron(II) gluconate | | | | | | | |
| Gon source) | Iron(II) citrate | 5 ppm | 5 ppm | 5 ppm | 5 ppm | 5 ppm | 5 ppm | 5 ppm |
| | Iron(II) sulfate | | | | | | | |
| | Iron(III) sulfate | | | | | | | |
| Other | Iron ascorbate complex | | | | | | | |
| components | Titanium oxide | | | | | | | |
| Solvent | Propylene glycol/water = 1/1 mixture | | | | | | | |
| | Purified water | Balance | Balance | Balance | Balance | Balance | Balance | Balance |
| Total | Total (%) | 100 | 100 | 100 | 100 | 100 | 100 | 100 |
| Evaluation | (1) Bactericidal activity after light | A | B | A | A | A | E | E |
| | irradiation | | | | | | | |
| | (2) Bactericidal activity before light | E | E | E | E | E | E | E |
| | irradiation | | | | | | | |
| | Colorability (color difference change ΔE) | 3.34 | 3.34 | 3.19 | 3.24 | 3.13 | 1.03 | 3.11 |

| | | Comparative | Example | Example | Example | Comparative |
|---|---|---|---|---|---|---|
| | | Example 8 | 11 | 12 | 13 | Example 9 |
| Compound | Ribuflavin | | | | | |
| (A) | Riboflavin sodium phosphate | 10 ppm | 10 ppm | 10 ppm | 10 ppm | |
| | Riboflavin butyrate | | | | | |
| | Lumichrome | | | | | |
| Sacrificial | Potassium L-ascorbate | | | | | |
| agent | Sodium L-ascorbate | | | | | |
| (B) | Calcium L-ascorbate | | | | | |
| | L-ascorbic acid | | | | | |
| | Sodium erythorbate | | | | | |
| | Triethanolamine | | | | | |
| | Triisopropanolamine | | | | | |
| | Sodium ferulate | | 20 ppm | | | |
| | Sodium tannate | | | 100 ppm | | |
| | (+)-Catechin sodium | | | | 50 ppm | 50 ppm |
| | (−)-Epigallocatechin sodium gallate | | | | | |
| | Glycerin | | | | | |
| | Polyglycerin | | | | | |
| | Xylitol | | | | | |
| Other | Alanine | | | | | |
| components | Sodium glutamate | 100 ppm | | | | |
| | Sodium itaconate | | | | | |
| | Sodium citrate | | | | | |
| Ion (C) | Iron(II) gluconate | | | | | |
| (ion source) | Iron(II) citrate | 50 ppm | 5 ppm | 5 ppm | 5 ppm | 5 ppm |
| | Iron(II) sulfate | | | | | |
| | Iron(III) sulfate | | | | | |
| Other | Iron ascorbate complex | | | | | |
| components | Titanium oxide | | | | | |
| Solvent | Propylene glycol/water = 1/1 mixture | | | | | |
| | Purified water | Balance | Balance | Balance | Balance | Balance |
| Total | Total (%) | 100 | 100 | 100 | 100 | 100 |
| Evaluation | (1) Bactericidal activity after light | E | B | B | B | E |
| | irradiation | | | | | |
| | (2) Bactericidal activity before light | E | E | E | E | E |
| | irradiation | | | | | |
| | Colorability (color difference change ΔE) | 3.38 | 4.17 | 7.02 | 6.03 | 6.32 |

TABLE 3

| | | | | | | Compar- | Compar- | | Compar- | Compar- |
|---|---|---|---|---|---|---|---|---|---|---|
| | | | | | | ative | ative | Ex- | ative | ative |
| | | Example | Example | Example | Example | Example | Example | ample | Example | Example |
| | | 14 | 15 | 16 | 17 | 10 | 11 | 18 | 12 | 13 |
| Compound | Riboflavin | | | | | | | | | |
| (A) | Riboflavin sodium | 10 ppm | 20 ppm | 20 ppm | 20 ppm | 10 ppm | 10 ppm | 10 ppm | 200 ppm | |
| | phosphate | | | | | | | | | |
| | Riboflavin butyrate | | | | | | | | | |
| | Lumichrome | | | | | | | | | |
| Sacrificial | Potassium L-ascorbate | | | | | | | 20 ppm | | |
| agent | Sodium L-ascorbate | | | | | | | | | |
| (B) | Calcium L-ascorbate | | | | | | | | | |
| | L-ascorbic acid | | | | | | | | | |
| | Sodium erythorbate | | | | | | | | | |
| | Triethanolamine | | | | | | | | | |
| | Triisopropanolamine | | | | | | | | | |
| | Sodium ferulate | | | | | | | 20 ppm | | |
| | Sodium tannate | | | | | | | | | |
| | (+)-Catechin sodium | | | | | | | | | |
| | (−)-Epigallocatechin | 100 ppm | | | | | | | | |
| | sodium gallate | | | | | | | | | |
| | Glycerin | | 1000 ppm | | | | | 20 ppm | | |
| | Polyglycerin | | | 1000 ppm | | | | | | |
| | Xylito | | | | 1000 ppm | | | | | |
| Other | Alanine | | | | | | | | | |
| components | Sodium glutamate | | | | | | | | | |
| | Sodium itaconate | | | | | 100 ppm | | | | |
| | Sodium citrate | | | | | | 100 ppm | | | |
| Ion (C) | Iron(Il) gluconate | | | | | | | 5 ppm | | |
| (ion source) | Iron(II) citrate | 5 ppm | 5 ppm | 5 ppm | 5 ppm | 5 ppm | 5 ppm | | | |
| | Iron(II) sulfate | | | | | | | | | |
| | Iron(III) sulfate | | | | | | | | | |
| Other | Iron ascorbate complex | | | | | | | | | |
| components | Titanium oxide | | | | | | | | | 100 ppm |
| Solvent | Propylene glycol/water = | | | | | | | | | |
| | 1/1 mixture | | | | | | | | | |
| | Purified water | Balance | Balance | Balance | Balance | Balance | Balance | Balance | Balance | Balance |
| Total | Total (%) | 100 | 100 | 100 | 100 | 100 | 100 | 100 | 100 | 101 |
| Evaluation | (1) Bactericidal activity | B | B | B | B | E | E | A | E | E |
| | after light irradiation | | | | | | | | | |
| | (2) Bactericidal activity | E | E | E | E | E | E | E | E | E |
| | before light irradiation | | | | | | | | | |
| | Colorability (color | 7.24 | 4.48 | 4.86 | 4.72 | 3.55 | 3.13 | 3.30 | 38.04 | 0.34 |
| | difference change ΔE) | | | | | | | | | |

### [Summary of Tables 1 to 3]

As shown in Comparative Example 3, the iron ascorbate complex of Patent Literature 1 failed to have a sufficient bactericidal effect with the present light source having a low photon flux density. As shown in Comparative Example 4, although a slight bactericidal effect can be obtained at a concentration of 1%, an application such as water in aquariums for breeding aquatic organisms is unrealistic because of the high concentration.

A solution such as Comparative Example 2 containing the compound (A) and not containing the sacrificial agent (B) failed to have a sufficient bactericidal effect. The present inventors have found that the riboflavin sodium phosphate set to 10,000 ppm exhibits a certain degree of bactericidal effect. However, as shown in the colorability evaluation of Comparative Example 12, it is found that a solution containing 200 ppm of riboflavin sodium phosphate is clearly colored, which becomes a problem when used for visual recognition of the inside of the solution.

As shown in Examples 1 to 18, it is found that the present photocatalyst composition containing the compound (A) and the sacrificial agent (B) can provide excellent bactericidal effect while suppressing the concentration of the compound (A).

### Examples 19 to 27

Photocatalyst composition solutions of Examples 19 to 27 were obtained in the same manner as in Example 1, except that the concentrations of each component and the blending amount in Example 1 were changed to those shown in Table 4. The obtained photocatalyst composition was evaluated for bactericidal properties and colorability by the same evaluation method as described above. The results are shown in Table 4.

TABLE 4

| | | Example | Example | Example | Example | Example | Example | Example | Example | Example |
|---|---|---|---|---|---|---|---|---|---|---|
| | | 19 | 20 | 21 | 22 | 23 | 24 | 25 | 26 | 27 |
| Compound | Riboflavin sodium | 10 ppm | 10 ppm | 10 ppm | 10 ppm | 10 ppm | 10 ppm | 10 ppm | 10 ppm | 10 ppm |
| (A) | phosphate | | | | | | | | | |
| Sacrificial | Sodium L-ascorbate | 20 ppm | 50 ppm | 100 ppm | 1000 ppm | 50000 ppm | 100 ppm | 100 ppm | 100 ppm | 100 ppm |
| agent | | | | | | | | | | |
| (B) | | | | | | | | | | |
| Ion (C) | Iron(II) sulfate | 5 ppm | 5 ppm | 5 ppm | 5 ppm | 5 ppm | 1 ppm | 0.1 ppm | 0.01 ppm | 0 ppm |
| (ion source) | | | | | | | | | | |
| Solvent | Purified water | Balance | Balance | Balance | Balance | Balance | Balance | Balance | Balance | Balance |
| Total | Total (%) | 100 | 100 | 100 | 100 | 100 | 100 | 100 | 100 | 100 |
| Evaluation | (1) Bactericidal activity | B | A | A | A | B | A | A | A | C |
| | after light irradiation | | | | | | | | | |
| | (2) Bactericidal activity | E | E | E | E | E | E | E | E | E |
| | before light irradiation | | | | | | | | | |
| | Color-difference meter | 3.55 | 3.57 | 3.56 | 3.19 | 3.91 | 3.55 | 3.64 | 3.55 | 3.59 |

As shown in Table 4, it is shown that the photocatalyst compositions of Examples 20 to 22 in which the mass ratio (A/B) of the compound (A) having an isoalloxazine skeleton and the like to the sacrificial agent (B) is 1/5 to 1/200 have particularly excellent photocatalytic effect.

In addition, it is shown that the photocatalyst compositions of Examples 24 to 26 in which the mass ratio (C/B) of the ion source of ions (C) to the sacrificial agent (B) is 1/100 to 1/10,000 have particularly excellent photocatalytic effect.

### <Evaluation of Persistence of Bactericidal Activity>

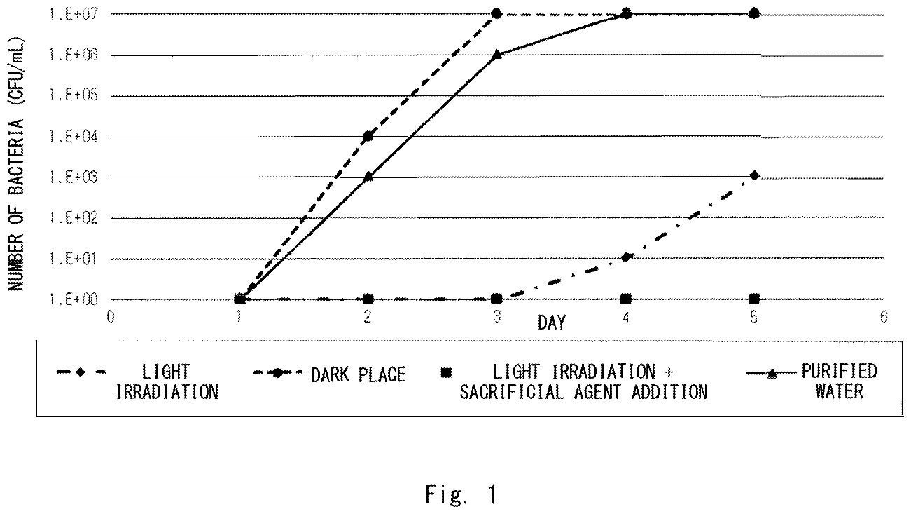

Three pieces of 100 ml of the photocatalyst composition solution of Example 2 and 100 ml of purified water for comparison were prepared, and 0.1 mL of the above inoculum solution adjusted to 1.0×10¹ CFU/ml was added every 24 hours, and the number of bacteria was measured immediately before adding the present inoculum solution. The photocatalyst composition solution of Example 2 was stored and operated under the following conditions (1) to (3). Purified water was stored under the following conditions (2). The results are shown in FIG. 1.
(1) Using a white LED (FHF32W2, manufactured by Sanyo Electric Co., Ltd.) as a light source at 25° C., irradiation was performed at a photon flux density of 20.25 μmol·m⁻²·s⁻¹.
(2) The sample was stored in a dark place at 25° C.
(3) Light irradiation was continued under the condition of (1), and sodium ascorbate equivalent to 20 ppm was added every 24 hours.

As shown in FIG. 1, the photocatalyst composition of Example 2 was able to suppress the increase in the number of bacteria for about 5 days. In addition, it is found that adding the sacrificial agent every 24 hours can maintain photocatalytic effect for a long time.

### Example 28

A polyurethane-based coating agent (RB6, manufactured by Yushiro Chemical Industry Co., Ltd.) was blended with riboflavin to have 100 ppm, sodium erythorbate to have 1000 ppm, and iron compounds to have 5 ppm to prepare a photocatalyst resin composition.

The photocatalyst resin composition was applied onto a petri dish and dried to form a coating film containing the catalyst composition.

A bacterial solution of 0.5 ml containing 1.0×10⁷ CFU/ml of bacteria was sprayed onto the obtained coating film. Then, irradiation of blue light with a wavelength of 450 nm and 1000 lx was performed for 2 hours.

The coating film after irradiation was subjected to stamping measurement using SAN-AI EZ-DipTTC (manufactured by Sanai Oil Co., Ltd.) to measure the number of bacteria.

In addition, as a control experiment, a polyurethane-based coating agent (RB6) was used instead of the above photocatalyst resin composition, and the number of bacteria was measured in the same manner as described above.

The number of bacteria on the coating film without the photocatalyst composition was approximately 1.0×10⁴ CFU/ml, whereas the number of bacteria on the coating film of Example 28 was less than 1.0×10³ CFU/ml. As described above, it is shown that the coating film obtained by combining the present photocatalyst composition with a binder resin can exhibit bactericidal effect.

### (Second Example Group)

The second example group shows the effect of the photocatalyst composition in which the sacrificial agent (B) contains one or more selected from chitosan and polyethyleneimine. In order to demonstrate the effect of long-term stability, photocatalyst compositions corresponding to the examples of the present invention are given as reference examples, but this does not deny the effect of the present invention. In addition, the first example group and the second example group are independent of each other, and the evaluation method and evaluation criteria are partly different.

### [Sacrificial Agent]

As a sacrificial agent, the following chitosans A to C, oligochitosan, and polyethyleneimine were used.
Chitosan A: Ichitosan M, manufactured by Dainichiseika Color & Chemicals Mfg. Co., Ltd. (Mp: 151,000, degree of deacetylation: about 85%)
Chitosan B: Chito Fresh (2), manufactured by Maruzen Pharmaceuticals Co., Ltd. (Mp: 79,000, degree of deacetylation: about 90%)
Chitosan C: Daichitosan PVL, manufactured by Dainichiseika Color & Chemicals Mfg. Co., Ltd. (Mp: 48,000, degree of deacetylation: about 85%)
Oligochitosan: COS-YS, manufactured by Yaizu Suisankagaku Industry Co., Ltd. (Mp: less than 6000)
Polyethyleneimine: Epomin P-1000, manufactured by Nippon Shokubai Co., Ltd. (Mp: 30,000)

The peak top molecular weight (Mp) is a value obtained as a standard pullulan conversion value by measurement under the following conditions with aqueous gel filtration chromatography (Chromaster (register trade mark), manufactured by Hitachi High-Tech Science Corporation).
Column: SB-805 HQ (gel filtration column, manufactured by Shodex (trade mark))
Mobile phase: 50 mM acetic acid, 0.3 M sodium nitrate aqueous solution
Column temperature: 40° C.

In addition, the degree of deacetylation was measured by a colloid titration method using polyvinyl potassium sulfate (PVSK).

### Preparation Example 1: Preparation of Chitosan a Aqueous Solution

The above chitosan A of 20 g was added to and dispersed in 1 L of pure water. (R)-2-hydroxypropionic acid (Tokyo Chemical Industry Co., Ltd.) of 7.5 g was added thereto and completely dissolved by stirring at room temperature for 3 hours to provide a chitosan A aqueous solution (20000 ppm).

### Preparation Example 2: Preparation of Chitosan B Aqueous Solution

The above chitosan B of 20 g was added to and dispersed in 1 L of pure water. D-glucuronic acid (Tokyo Chemical Industry Co., Ltd.) of 25.8 g was added thereto and completely dissolved by stirring at room temperature for 3 hours to provide a chitosan B aqueous solution (20000 ppm).

### Preparation Example 3: Preparation of Chitosan C Aqueous Solution

The above chitosan C of 20 g was added to and dispersed in 1 L of pure water. Malic acid (Tokyo Chemical Industry Co., Ltd.) of 16.5 g was added thereto and completely dissolved by stirring at room temperature for 3 hours to provide a chitosan C aqueous solution (20000 ppm).

### Preparation Example 4: Preparation of Oligochitosan Aqueous Solution

The above oligochitosan of 20 g was added to 1 L of pure water and completely dissolved by stirring at room temperature for 3 hours to provide an oligochitosan aqueous solution (20000 ppm).

### Preparation Example 5: Preparation of Polyethyleneimine Aqueous Solution

The above polyethyleneimine of 20 g was added to and dispersed in 1 L of pure water. (R)-2-hydroxypropionic acid (Tokyo Chemical Industry Co., Ltd.) of 38.3 g was added thereto and completely dissolved by stirring at room temperature for 1 hours to provide a polyethyleneimine aqueous solution (20000 ppm).

### Example 31: Preparation of Photocatalyst Composition Solution

Riboflavin equivalent to 5 ppm, 10% of the chitosan A aqueous solution (equivalent to 2000 ppm of chitosan A), and 1 ppm equivalent of iron (II) gluconate were mixed, then water was added so as to prepare the composition described in Table 1, and a photocatalyst composition solution of Example 31 was obtained.

### Examples 32 to 56: Preparation of Photocatalyst Composition Solution

Photocatalyst composition solutions of Examples 32 to 56 were obtained in the same manner as in Example 31, except that the concentrations of each component and the blending amount in Example 31 were changed to those shown in Tables 5 to 7.

### Comparative Example 31, Comparative Examples 33 to 37, and Reference Example 32: Preparation of Solution

The solutions of Comparative Example 31, Comparative Examples 33 to 37, and Reference Example 32 were obtained in the same manner as in Example 31, except that the concentrations of each component and the blending amount in Example 31 were changed to those shown in Tables 5 to 7.

### [Evaluation]

### <Photobactericidal Activity Evaluation>

### (1) Preparation of Test Solution

Escherichia coli K-12 strain (NBRC3301) was used as a test strain. One colony was taken with a platinum loop from a normal bouillon agar medium (Eiken (register trade mark), manufactured by Eiken Chemical Co., Ltd.) in which the test bacteria was cultured, added to 10 mL liquid medium (autoclave sterilized) in a test tube, cultured for 2 days in an incubator set at 30° C., and adjusted to the number of bacteria of 4 to 6×10⁹ CFU/ml. The resulting bacterial solutions were each diluted 1000-fold with the photocatalyst composition of Example 1 to prepare test solutions with the number of bacteria of 4 to 6×10⁶ CFU/ml. The photocatalyst composition was sterilized by filtration using Millex for sterilization having a hole diameter of 0.22 μm immediately before the test.

### (2) Preparation of Test Plate

A sterilized filter paper for humidity control was placed on the bottom of a sterilized petri dish, 4.5 mL of sterilized water was added thereto, and a U-shaped tube was placed therein so that the test plate and the filter paper for humidity control did not touch each other. A sterilized glass plate (5.5 cm×5.5 cm) was placed thereon, and a sterilized cotton standard cloth (defined in JIS L 0803) was placed thereon. The cotton standard cloth was inoculated with 0.2 mL of the test solution prepared in (1), and was covered with contact glass (borosilicate glass: 5.5 cm×5.5 cm). Furthermore, a moisturizing glass plate (borosilicate glass (10 cm×10 cm)) was placed on the petri dish. Two or more pieces of the same test plates were made.

Test plates were prepared in the same manner for the photocatalyst compositions of other examples and comparative examples.

### (3) Light Irradiation and Measurement

One of the prepared test plates was left under light irradiation (light source: white LED, FHF32W2 manufactured by Sanyo Electric Co., Ltd.) at 25° C. for 4 hours. The light irradiation illuminance through the moisturizing glass and the adhesion glass was measured previously with an illuminometer (illuminometer T-10A, manufactured by Konica Minolta, Inc.), and the photon flux density was adjusted to 50 μmol/m⁻²/s⁻¹.

Another test plate was left in a dark place at 25° C. for 4 hours.

Each of the test plates was transferred to a sterilized stomacher bag, 20 mL of washing solution (sterilized water) was added thereto (100-fold dilution), bacteria were washed out from the test sample, and the number of bacteria was measured by a 10-fold dilution method.

### (Evaluation Criteria)

The ratio of the bacteria number ((1)/(2)) between the number of bacteria after light irradiation (1) and the number of bacteria after dark storage (2) was calculated and evaluated.
A (outstanding): The ratio of the bacteria number was less than 1/100.
B (excellent): The ratio of the bacteria number was 1/100 or more and less than 1/10.
C (good): The ratio of the bacteria number was 1/10 or more and less than 1/4.
D (fair): The ratio of the bacteria number was 1/4 or more and less than 1/2.
E (bad): The ratio of the bacteria number was 1/2 or more.

### <Colorability Evaluation>

An evaluation sample was obtained in such a manner that a predetermined amount of each photocatalyst composition obtained in Examples and Comparative Examples in a spray bottle was sprayed on a standard test cloth cut to 5 cm×5 cm from a position 10 cm away, and the color difference represented by the following formula before and after coating was measured. The results are shown in Tables 5 to 7.

Standard cloth: Crecia Techno Wipe C100-M, manufactured by Nippon Paper Crecia Co., Ltd.

Color Difference ΔE=[(ΔL)²+(Δa)²+(Δb)²]^{1/2}
in which ΔL=L value (after coating)−L value (before coating)
Δa=a value (after coating)−a value (before coating)
Δb=b value (after coating)−b value (before coating)

### (Evaluation Criteria)

A (excellent): ΔE was less than 10.0.
B (good): ΔE was 10.0 or more and less than 20.0.
E (bad): ΔE was 20.0 or more.

### <Stability Evaluation of Photobactericidal Activity>

The photocatalyst compositions of each Example and each Comparative Fxample were stored in a constant temperature bath at 50° C. for one month. The photocatalyst composition after storage was evaluated in the same manner as the photobactericidal activity evaluation, and the ratio of the bacteria number ((1)/(2)) was calculated. The ratio ((A)/(B)×100) between the ratio of the bacteria number of the photocatalyst composition after storage (A) and the ratio of the bacteria number of the photobactericidal activity evaluation (B) was calculated, and evaluated according to the following criteria. The results are shown in Tables 5 to 7.

### (Evaluation Criteria)

A (excellent): (A)/(B) was 98% or more.
B (good): (A)/(B) was 50% or more and less than 98%.
C (fair): (A)/(B) was less than 50%.

### [Table 5]

TABLE 5

| | | Example | Example | Example | Example | Example | Example | Example |
|---|---|---|---|---|---|---|---|---|
| | | 31 | 32 | 33 | 34 | 35 | 36 | 37 |
| Compound | Riboflavin | 5 ppm | | | | 10 ppm | 10 ppm | 10 ppm |
| (A) | Riboflavin sodium phosphate | | 5 ppm | | | | | |
| | Riboflavin butyrate | | | 5 ppm | | | | |
| | Lumichrome | | | | 5 ppm | | | |
| Sacrificial | Chitosan A | 2000 ppm | 1000 ppm | 400 ppm | 600 ppm | 1000 ppm | 2000 ppm | 2000 ppm |
| agent | Chitosan B | | | | | | | |
| (B) | Chitosan C | | | | | | | |
| | Oligochitosan | | | | | | | |
| | Polyethyleneimine | | | | | | | |
| Other | Glucosamine | | | | | | | |
| components | | | | | | | | |
| Sacrificial agent | Sodium L-ascorbate | | | | | | | |
| (Reference | | | | | | | | |
| Example) | | | | | | | | |
| lon (C) | Iron(II) gluconate | 1 ppm | | | | | | |
| (ion source) | Iron(II) citrate | | 1 ppm | | | | | |
| | Iron(II) sulfate | | | 1 ppm | 1 ppm | | | |
| | Iron(III) sulfate | | | | | 1 ppm | | |
| | Copper(II) gluconate | | | | | | 1 ppm | |
| | Nickel(II) sulfate | | | | | | | 1 ppm |
| Other | Naphthalene-mPEG(500) | | | | | | | |
| components | carbamate conjugate | | | | | | | |
| | Titanium oxide | | | | | | | |
| Solvent | Propylene glycol/water = 1/1 mixture | | | | | | | |
| | Purified water | Balance | Balance | Balance | Balance | Balance | Balance | Balance |
| Total | Total (%) | 100 | 100 | 100 | 100 | 100 | 100 | 100 |
| Evaluation | Photobactericidal activity evaluation | A | A | A | A | B | B | C |
| | Colorability | A | A | A | A | A | A | A |
| | (color difference change ΔE) | | | | | | | |
| | Photobactericidal activity in 50° C. | A | A | A | A | A | A | A |
| | constant temperature bath for 1 month | | | | | | | |

| | | Example | Example | Example | Comparative | Reference |
|---|---|---|---|---|---|---|
| | | 38 | 39 | 40 | Example | Example |
| Compound | Riboflavin | 5 ppm | 5 ppm | 10 ppm | 10 ppm | 10 ppm |
| (A) | Riboflavin sodium phosphate | | | | | |
| | Riboflavin butyrate | | | | | |
| | Lumichrome | | | | | |
| Sacrificial | Chitosan A | | | | | |
| agent | Chitosan B | 1000 ppm | | | | |
| (B) | Chitosan C | | 1000 ppm | | | |
| | Oligochitosan | | | 1000 ppm | | |
| | Polyethyleneimine | | | | | |
| Other | Glucosamine | | | | 1000 ppm | |
| components | | | | | | |
| Sacrificial agent | Sodium L-ascorbate | | | | | 1000 ppm |
| (Reference | | | | | | |
| Example) | | | | | | |
| lon (C) | Iron(II) gluconate | 1 ppm | 1 ppm | 1 ppm | 1 ppm | 1 ppm |
| (ion source) | Iron(II) citrate | | | | | |
| | Iron(II) sulfate | | | | | |
| | Iron(III) sulfate | | | | | |
| | Copper(II) gluconate | | | | | |
| | Nickel(II) sulfate | | | | | |
| Other | Naphthalene-mPEG(500) | | | | | |
| components | carbamate conjugate | | | | | |
| | Titanium oxide | | | | | |
| Solvent | Propylene glycol/water = 1/1 mixture | | | | | |
| | Purified water | Balance | Balance | Balance | Balance | Balance |
| Total | Total (%) | 100 | 100 | 100 | 100 | 100 |
| Evaluation | Photobactericidal activity evaluation | A | A | D | E | A |
| | Colorability | A | A | A | A | A |
| | (color difference change ΔE) | | | | | |
| | Photobactericidal activity in 50° C. | A | A | A | A | C |
| | constant temperature bath for 1 month | | | | | |

TABLE 6

| | | Ex- | Ex- | Compar- | Compar- | Compar- | Ex- | Ex- | Ex- | Ex- | Ex- |
|---|---|---|---|---|---|---|---|---|---|---|---|
| | | ample | ample | ative | ative | ative | ample | ample | ample | ample | ample |
| | | 41 | 42 | Example | Example | Example | 43 | 44 | 45 | 46 | 47 |
| Compound | Riboflavin | 10 ppm | 10 ppm | | 10 ppm | 200 ppm | 5 ppm | 5 ppm | 5 ppm | 2 ppm | 5 ppm |
| (A) | Riboflavin sodium | | | | | | | | | | |
| | phosphate | | | | | | | | | | |
| | Riboflavin butyrate | | | | | | | | | | |
| | Lumichrome | | | | | | | | | | |
| Sacrificial | Chitosan A | | 2000 ppm | 2000 ppm | | | 19980 ppm | 100 ppm | 40 ppm | 10 ppm | 100 ppm |
| agent | Chitosan B | | | | | | | | | | |
| (B) | Chitosan C | | | | | | | | | | |
| | Oligochitosan | | | | | | | | | | |
| | Polyethyleneimine | 20 ppm | | | | | | | | | |
| Other | Glucosamine | | | | | | | | | | |
| components | | | | | | | | | | | |
| Sacrificial | Sodium L-ascorbate | | | | | | | | | | |
| agent | | | | | | | | | | | |
| (Reference | | | | | | | | | | | |
| Example) | | | | | | | | | | | |
| Ion (C) | Iron(II) gluconate | 1 ppm | | 1 ppm | 1 ppm | | 1 ppm | 1 ppm | 1 ppm | 1 ppm | 0.01 ppm |
| (ion source) | Iron(II) citrate | | | | | | | | | | |
| | Iron(II) sulfate | | | | | | | | | | |
| | fron(III) sulfate | | | | | | | | | | |
| | Copper(II) gluconate | | | | | | | | | | |
| | Nickel(II) sulfate | | | | | | | | | | |
| Other | Naphthalene-mPEG | | | | | | | | | | |
| | (500) carbamate | | | | | | | | | | |
| | conjugate | | | | | | | | | | |
| components | Titanium oxide | | | | | | | | | | |
| Solvent | Propylene glycol/ | | | | | | | | | | |
| | water = 1/1 mixture | | | | | | | | | | |
| | Purified water | Balance | Balance | Balance | Balance | Balance | Balance | Balance | Balance | Balance | Balance |
| Total | Total (%) | 100 | 100 | 100 | 100 | 100 | 100 | 100 | 100 | 100 | 100 |
| Evaluation | Photobactericidal | A | D | E | E | E | D | A | B | D | C |
| | activity evaluation | | | | | | | | | | |
| | Colorability | A | A | A | A | E | A | A | A | A | A |
| | (color difference | | | | | | | | | | |
| | change ΔE) | | | | | | | | | | |
| | Photobactericidal | A | A | A | A | A | A | A | A | A | A |
| | activity in 50° C. | | | | | | | | | | |
| | constant temperature | | | | | | | | | | |
| | bath for 1 month | | | | | | | | | | |

TABLE 7

| | | Example | Example | Example | Example | Example | Example | Example |
|---|---|---|---|---|---|---|---|---|
| | | 48 | 49 | 50 | 51 | 52 | 53 | 54 |
| Compound | Riboflavin | 1 ppm | 1 ppm | 5 ppm | 10 ppm | 2 ppm | 50 ppm | 5 ppm |
| (A) | Riboflavin sodium phosphate | | | | | | | |
| | Riboflavin butyrate | | | | | | | |
| | Lumichrome | | | | | | | |
| Sacrificial | Chitosan A | | | | | | 4000 ppm | 400 ppm |
| agent | Chitosan B | | | | | | | |
| (8) | Chitosan C | | | | | | | |
| | Oligochitosan | | | | | | | |
| | Polyethyleneimine | 19980 ppm | 10000 ppm | 1000 ppm | 80 ppm | 10 ppm | | |
| Other | Glucosamine | | | | | | | |
| components | | | | | | | | |
| Sacrificial agent | Sodium L-ascorbate | | | | | | | |
| (Reference | | | | | | | | |
| Example) | | | | | | | | |
| Ion (C) | Iron(II) gluconate | 1 ppm | 10 ppm | 5 ppm | 10 ppm | 1 ppm | 1 ppm | 1 ppm |
| (ion source) | Iron(II) citrate | | | | | | | |
| | Iron(II) sulfate | | | | | | | |
| | Iron(III) sulfate | | | | | | | |
| | Copper(II) gluconate | | | | | | | |
| | Nickel(II) sulfate | | | | | | | |
| Other | Naphthalene-mPEG(500) | | | | | | | |
| components | carbamate conjugate | | | | | | | |
| | Titanium oxide | | | | | | | |
| Solvent | Propylene glycol/water = 1/1 mixture | | | | | | | |
| | Purified water | Balance | Balance | Balance | Balance | Balance | Balance | Balance |
| Total | Total (%) | 100 | 100 | 100 | 100 | 100 | 100 | 100 |
| Evaluation | Photobactericidal activity evaluation | D | B | A | B | D | A | A |
| | Colorability | A | A | A | A | A | 8 | A |
| | (color difference change ΔE) | | | | | | | |
| | Photobactericidal activity in 50° C. | A | A | A | A | A | A | A |
| | constant temperature bath for 1 month | | | | | | | |

| | | | |
|---|---|---|---|
| Example | Example | Comparative | Comparative |
| 55 | 56 | Example | Example |

| | | | | | |
|---|---|---|---|---|---|
| Compound | Riboflavin | 1 ppm | 0.5 ppm | | |
| (A) | Riboflavin sodium phosphate | | | | |
| | Riboflavin butyrate | | | | |
| | Lumichrome | | | | |
| Sacrificial | Chitosan A | 400 ppm | 400 ppm | | |
| agent | Chitosan 8 | | | | |
| (8) | Chitosan C | | | | |
| | Oligochitosan | | | | |
| | Polyethyleneimine | | | | |
| Other | Glucosamine | | | | |
| components | | | | | |
| Sacrificial agent | Sodium L-ascorbate | | | | |
| (Reference | | | | | |
| Example) | | | | | |
| Ion (C) | Iron(II) gluconate | 1 ppm | 1 ppm | | |
| (ion source) | Iron(II) citrate | | | | |
| | Iron(II) sulfate | | | | |
| | Iron(III) sulfate | | | | |
| | Copper(II) gluconate | | | | |
| | Nickel(II) sulfate | | | | |
| Other | Naphthalene-mPEG(500) | | | 100 ppm | |
| components | carbamate conjugate | | | | |
| | Titanium oxide | | | | 100 ppm |
| Solvent | Propylene glycol/water = 1/1 mixture | | | | |
| | Purified water | Balance | Balance | Balance | Balance |
| Total | Total (%) | 100 | 100 | 100 | 100 |
| Evaluation | Photobactericidal activity evaluation | C | D | E | E |
| | Colorability | A | A | A | A |
| | (color difference change ΔE) | | | | |
| | Photobactericidal activity in 50° C. | A | A | A | A |
| | constant temperature bath for 1 month | | | | |

### [Summary of Tables 5 to 7]

As shown in Comparative Example 31, the photocatalyst composition using glucosamine that is a monomer of chitosan had insufficient photobactericidal activity. In addition, the photocatalyst composition of Reference Example 32 using sodium L-ascorbate showed a decrease in photobactericidal activity during one month.

As shown in Examples 31 to 58, it is found that the present photocatalyst composition containing a compound having an isoalloxazine skeleton or an alloxazine skeleton, and one or more selected from chitosan and polyethyleneimine exhibits excellent bactericidal effect and long-term stability of photobactericidal activity while suppressing the concentration of the compound having an isoalloxazine skeleton and the like.

### Examples 57 to 67

Photocatalyst composition solutions of Examples 57 to 67 were obtained in the same manner as in Example 31, except that each of the component and the blending amount in Example 31 was changed to the formulation shown in Table 8. The numerical value of each component in Table 8 indicates the mass ratio based on the total of each component being 100% by mass.

The obtained photocatalyst composition was evaluated for photobactericidal activity, colorability, and photobactericidal activity stability by the same evaluation method as described above. The results are shown in Table 8.

TABLE 8

| | | Example 57 | Example 58 | Example 59 | Example 60 | Example 61 | Example 62 | Example 63 |
|---|---|---|---|---|---|---|---|---|
| Compound | Riboflavin | 0.025 | 4.7615 | 1.232 | 4.717 | 10.870 | 15.385 | 0.005 |
| (A) | | | | | | | | |
| Sacrificial | Chitosan A | 99.970 | 95.2290 | 98.522 | 94.340 | 86.956 | 76.923 | |
| agent | Polyethyleneimine | | | | | | | 99.990 |
| (B) | | | | | | | | |
| Ion (C) | Iron(II) gluconate | 0.005 | 0.0095 | 0.246 | 0.943 | 2.174 | 7.692 | 0.005 |
| (ion source) | | | | | | | | |
| Total | Total (%) | 100.000 | 100.000 | 100.000 | 100.000 | 100.000 | 100.000 | 100.000 |
| Evaluation | Photobactericidal activity evaluation | D | C | A | A | B | B | D |
| | Colorability | A | A | A | A | A | A | A |
| | (color difference change ΔE) | | | | | | | |
| | Photobactericidal activity in 50° C. | A | A | A | A | A | A | A |
| | constant temperature bath for 1 | | | | | | | |
| | | | | | Example 64 | Example 65 | Example 66 | Example 67 |

| | | | | | |
|---|---|---|---|---|---|
| Compound | Riboflavin | 0.010 | 0.495 | 10.989 | 15.385 |
| (A) | | | | | |
| Sacrificial | Chitosan A | | | | |
| agent | Polyethyleneimine | 99.890 | 99.010 | 87.912 | 76.923 |
| (B) | | | | | |
| lon (C) | Iron(II) gluconate | 0.100 | 0.495 | 1.099 | 7.692 |
| (ion source) | | | | | |
| Total | Total (%) | 100.000 | 100.00 | 100.00 | 100.00 |
| Evaluation | Photobactericidal activity evaluation | B | A | B | D |
| | Colorability | A | A | A | A |
| | (color difference change ΔE) | | | | |
| | Photobactericidal activity in 50° C. | A | A | A | A |
| | constant temperature bath for 1 | | | | |

As shown in Table 8, a particularly excellent photobactericidal activity was exhibited when the mass ratio (C/B) of the mass of the metal ion source (C) to the mass of the sacrificial agent (B) was in the range of 1/10000 to 1/20.

### Example 68

A polyurethane-based coating agent (RB6, manufactured by Yushiro Chemical Industry Co., Ltd.) was blended with riboflavin to have 10 ppm, chitosan A aqueous solution to have 1%, and iron (II) gluconate to have 1.4 ppm to prepare a photocatalyst resin composition. The photocatalyst resin composition was applied onto a petri dish and dried to form a coating film containing the catalyst composition.

On the resulting coating film, 0.5 ml of Escherichia coli K-12 strain (NBRC3301) solution containing 8.3×10⁷ CFU/ml of bacteria was applied by spraying. Then, the coating film was irradiated with blue light having a wavelength of 450 nm and a photon flux density of 50 μmol m⁻²·s⁻¹ for 3 hours.

The coating film after irradiation was subjected to stamping measurement using SAN-AI EZ-DipTTC (manufactured by Sanai Oil Co., Ltd.) to measure the number of bacteria.

In addition, as a control experiment, a polyurethane-based coating agent (RB6) was used instead of the photocatalyst resin composition, and the number of bacteria was measured in the same manner as described above.

The number of bacteria on the coating film without the photocatalyst composition was approximately 1.0×10⁵, whereas the number of bacteria on the coating film of Example 68 was <1.0×10¹ or less. As described above, it is shown that the coating film obtained by combining the present photocatalyst composition with a binder resin can exhibit bactericidal effect.

This application is based upon and claims the benefit of priority from Japanese Patent Application No. 2020-174034 filed on Oct. 15, 2020 and Japanese Patent Application No. 2021-130590 filed on Aug. 10, 2021, the entire disclosures of which are incorporated herein in its entirety by reference.

1. A photocatalyst composition comprising:
a compound (A) having an isoalloxazine skeleton or an alloxazine skeleton; and
a sacrificial agent (B).

2. The photocatalyst composition according to claim 1, wherein the compound (A) having an isoalloxazine skeleton or an alloxazine skeleton includes a compound represented by a general formula (1) below or a compound represented by a general formula (2) below: 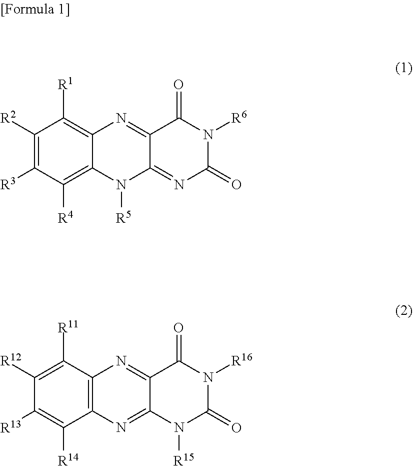
wherein, in the formula (1),
R¹, R², R³, and R⁴ each independently represent a hydrogen atom, a halogen atom, or a hydrocarbon group optionally having a substituent, and
R⁵ and R⁶ each independently represent a hydrogen atom or a hydrocarbon group optionally having a substituent, and
wherein, in the formula (2),
R¹¹, R¹², R¹³, and R¹⁴ each independently represent a hydrogen atom, a halogen atom, or a hydrocarbon group optionally having a substituent, and
R¹⁵ and R¹⁶ each independently represent a hydrogen atom or a hydrocarbon group optionally having a substituent.

3. The photocatalyst composition according to claim 1, wherein the compound (A) having an isoalloxazine skeleton or an alloxazine skeleton includes one or more selected from riboflavin and riboflavin derivatives.

4. The photocatalyst composition according to claim 1, wherein the sacrificial agent (B) includes one or more selected from ascorbic acid compounds, polyols, polyphenols, and alkanolamines.

5. The photocatalyst composition according to claim 1, wherein the sacrificial agent (B) includes one or more selected from chitosan and polyethyleneimine.

6. The photocatalyst composition according to claim 5, wherein the chitosan has a degree of deacetylation of 50% or more.

7. The photocatalyst composition according to claim 5, wherein the chitosan has a molecular weight of 5,000 to 1,000,000.

8. The photocatalyst composition according to claim 1, further comprising one or more ions (C) selected from iron ions, copper ions, and nickel ions.

9. The photocatalyst composition according to claim 1, wherein a mass ratio (A/B) of the compound (A) having an isoalloxazine skeleton or an alloxazine skeleton to the sacrificial agent (B) is 1/2 to 1/10000.

10. A photocatalyst composition solution comprising:
the photocatalyst composition according to any one of claim 1; and
a solvent.

11. A photocatalyst member comprising:
a coating film comprising the photocatalyst composition according to claim 1 on a base material.

12. A method for using a photocatalyst composition, comprising:
supplying a sacrificial agent (B) to
(I) a composition solution comprising a compound (A) having an isoalloxazine skeleton or an alloxazine skeleton and a solvent, or
(II) a member comprising a coating film comprising the compound (A) having an isoalloxazine skeleton or an alloxazine skeleton on a base material.

13. A space disinfection method comprising:
spraying the photocatalyst composition according to claim 1 into a space.
